(19) United States
(12) Patent Application Publication
(10) Pub. No.: US 2024/0342158 A1
(43) Pub. Date: Oct. 17, 2024

(54) METHOD OF TREATING AND/OR PREVENTING TYPE 1 DIABETES WITH CEPHARANTHINE

(71) Applicant: ALBERT EINSTEIN COLLEGE OF MEDICINE, Bronx, NY (US)

(72) Inventors: Yaron TOMER, Chappaqua, NY (US); Cheuk Wun LI, Brooklyn, NY (US)

(21) Appl. No.: 18/294,459

(22) PCT Filed: Aug. 1, 2022
(86) PCT No.: PCT/US2022/039029

§ 371 Date: Feb. 1, 2024

Related U.S. Application Data
(60) Provisional application No. 63/228,381, filed on Aug. 2, 2021.

(51) Int. Cl. A61K 31/4741; A61K 38/28; A61P 3/10

(52) U.S. Cl. CPC A61K 31/4741; A61K 38/28; A61P 3/10

(57) ABSTRACT

This disclosure provides methods of treating and/or preventing type 1 diabetes (T1D) and/or a syndrome including type 1 diabetes and autoimmune thyroiditis in a subject. The methods include administering a therapeutically effective amount of cepharanthine or a pharmaceutically acceptable salt, solvate or derivative thereof to the subject, wherein the subject has a human leucocyte antigen class II, DR3 allele (HLA-DR3) genotype.

### CROSS-REFERENCE TO RELATED APPLICATIONS

This application claims priority to U.S. Provisional Application 63/228,381 filed on Aug. 22, 2021, which is incorporated herein by reference in its entirety.

### FEDERAL RESEARCH STATEMENT

This invention was made with government support under grant number DK067555 awarded by the National Institute of Health—National Institute of Diabetes and Digestive and Kidney Diseases (NIH-NIDDK). The government has certain rights in the invention.

### BACKGROUND

Type 1 diabetes (T1D) is caused by a T-cell attack on the pancreatic beta cells that secrete insulin. Type 1 diabetes (T1D) is a chronic, autoimmune disorder in which the insulin producing pancreatic beta cells are destroyed by an auto-immune attack. This results in insulin deficiency and hyperglycemia leading to the associated long-term complications. Every year approximately 64,000 new patients are diagnosed with T1D in the US, and the prevalence of T1D in the US is approximately 1.4 million. Moreover, the incidence of T1D has been increasing since World War II at alarming rates. The treatment of T1D has not changed significantly over the past century and is still based on lifelong insulin replacement, which is both challenging and insufficient. The main advances in the treatment of T1D were the development of modified insulins with better kinetic properties and the design of improved delivery methods of insulin such as insulin pumps. However, despite the development of newer insulins and advancements in diabetes technology the majority of T1D patients do not achieve the American Diabetes Association (ADA) glycemic targets, and even patients that achieve the ADA target of HbA1c≤6.9% had double mortality from any cause compared to matched controls. Additionally, despite decades of research, there is no curative/preventive therapy for T1D that can reverse/prevent the autoimmune response causing beta cell destruction.

Recently, several new immune therapies have shown promise in delaying the onset of disease in individuals at high risk or slowing progression in patients with new onset T1D. However, all of these therapeutic approaches are not targeted and are associated with global immune suppression and significant side effects. Therefore, there is an unmet need to develop targeted immune therapies that can reverse/prevent the autoimmune attack on the beta cells.

### SUMMARY

The disclosure provides a method of treating and/or preventing type 1 diabetes (T1D) in a subject in need thereof comprising administering to the subject a therapeutically effective amount of cepharanthine or a pharmaceutically acceptable salt, solvate, or derivative thereof.

The disclosure provides a method of treating and/or preventing type 1 diabetes (T1D) in a subject comprising administering a therapeutically effective amount of cepharanthine or a pharmaceutically acceptable salt, solvate, or derivative thereof to the subject, wherein the subject has a human leucocyte antigen class II, DR3 allele (HLA-DR3) genotype.

The disclosure provides cepharanthine or a pharmaceutically acceptable salt, solvate, or derivative thereof to the subject for use in treating T1D in a subject, including in a subject having a human leucocyte antigen class II, DR3 allele (HLA-DR3) genotype.

The disclosure provides the use of cepharanthine or a pharmaceutically acceptable salt, solvate, or derivative thereof in the manufacture of a medicament for the treatment of T1D in a subject, including in a subject having a human leucocyte antigen class II, DR3 allele (HLA-DR3) genotype.

The disclosure also provides a method of treating and/or preventing autoimmune polyglandular syndrome type 3 variant (defined as the presence of T1D and autoimmune thyroiditis in the same person) in a subject comprising administering a therapeutically effective amount of cepharanthine or a pharmaceutically acceptable salt, solvate, or derivative thereof to the subject, including a subject that has a human leucocyte antigen class II, DR3 allele (HLA-DR3) genotype.

The disclosure provides cepharanthine or a pharmaceutically acceptable salt, solvate, or derivative thereof to the subject for use in treating and/or preventing autoimmune polyglandular syndrome type 3 variant in a subject, including in a subject having a leucocyte antigen class II, DR3 allele (HLA-DR3) genotype.

The disclosure provides the use of cepharanthine or a pharmaceutically acceptable salt, solvate, or derivative thereof in the manufacture of a medicament for use in treating and/or preventing autoimmune polyglandular syndrome type 3 variant in a subject, including in a subject having a leucocyte antigen class II, DR3 allele (HLA-DR3) genotype.

The disclosure provides a method of treating and/or preventing type 1 diabetes in a subject having type 1 diabetes or having a high risk for type 1 diabetes, the method comprising: determining that the subject has a human leucocyte antigen class II, DR3 allele (HLA-DR3) genotype; and administering a therapeutically effective amount of cepharanthine, or a pharmaceutically acceptable salt, solvate, or derivative thereof to the subject.

The disclosure includes methods and uses in which cepharanthine is the only active agent and methods and uses in which cepharanthine is used in combination with another active agent such as anti-thymocyte globulin, insulin, an amylinomimetic drug, or a combination thereof.

### BRIEF DESCRIPTION OF THE DRAWINGS

FIG. 1: A representative structure of the interaction between human leucocyte antigen-DR3 (HLA-DR3) and thyroid peroxidase-758 (TPO.758), one of the 3 unique autoimmune polyglandular syndrome 3 variant (APS3v) peptides. The binding pockets P1, P4, P6 and P9 are highlighted. P1 is filled with the residue Leu and P9 with Tyr. Pockets P4, P5 and P6 create a polar arrangement characterized by positive potential from Arg-β74 facing negative potentials from the opposing peptide residues.

FIG. 2A-FIG. 2D: Four compounds were tested in an ELISA inhibition assay at a final concentration of 0.4 millimolar (mM). Three compounds (S5 (FIG. 2B), S15 (FIG. 2C), S53 (FIG. 2D)) showed significant inhibition of binding for all 3 peptides to the HLA-DR3 pocket. Compound S9 (FIG. 2A) only blocked GAD.492 and Tg.1571 binding.

FIGS. 3A-D: S53 at decreasing concentrations (0.4 mM, 0.2 mM, 0.1 mM, 0.5 mM) was tested for blocking the binding of biotinylated APO, TPO.758, GAD.492 and Tg.1571 to APS3v-HLA-DR3 by ELISA. The ELISA results shows that S53 blocked biotinylated APO (FIG. 3A), TPO.758 (FIG. 3B), GAD.492 (FIG. 3C) and Tg.1571 (FIG. 3D) in a dose-dependent manner.

FIG. 4: DR3 mice (carrying the APS3v-DR3 pocket signature) were immunized with the 4 peptides that elicited strong T-cell responses (data not shown). Antibody responses were measured in a peptide antibody ELISA. Three of the peptides induced strong antibody responses in the immunized mice, Tg.1571, TPO.758, GAD.492, suggesting that they are major T-cell epitopes in APS3v.

FIG. 5A-FIG. 5D: NOD-DR3 mice (n=22) were immunized with the 3 peptides shown to be major APS3v epitopes (Tg.1571, TPO.758, GAD.492). Mice were sacrificed and their splenocytes were stimulated with the 3 peptides used for immunization. T-cell activation was measured by the production of interferon gamma (IFNγ). All 3 peptides elicited strong T-cell activation (positive control—CD3/CD28 beads; NC—negative control). Cytokine production and autoantibody response of humanized NOD-DR3 mice co-immunized with Tg.1571, GAD.492 and TPO.758. Interferon gamma production (FIG. 5A) and autoantibody response to Tg.1571 (FIG. 5B), GAD.492 (FIG. 5C) and TPO.758 (FIG. 5D) of humanized NOD-DR3 mice co-immunized with the three thyroid and islet peptides. NC, negative control peptide. Anti-CD3/CD28 beads, position control. Control mice were immunized with PBS and adjuvant. *, p<0.05; **, p<0.01; ***, p<0.001.

FIG. 6A and FIG. 6B: Clinical manifestation of AITD in humanized NOD-DR3 mice co-immunized with Tg.1571, GAD.492 and TPO.758. Autoantibody response to mouse Tg (FIG. 6A) and free T4 levels (FIG. 6B) of humanized NOD-DR3 mice co-immunized with the three thyroid and islet peptides, compared to control mice immunized with PBS and adjuvant. *, p<0.05; ***, p<0.001.

FIG. 7A-FIG. 7F: NOD-DR3 mice (n=8) were immunized with the 3-peptide cocktail (Tg.1571, TPO.758, GAD.492). Mice were sacrificed and their splenocytes were stimulated with the peptides used for immunization with or without molecules S5, S9, S15, and S53 at a concentration of 1 micromolar (p M). (FIG. 7A, B) Tg.1571; (FIG. 7C, D) GAD.492, (FIG. 7E, F) TPO.758. As shown only S53 (cepharanthine) consistently inhibited T-cell activation to all 3 peptides as demonstrated by significantly decreased production of IFNγ and IL-2. Percent inhibition by S53 (cepharanthine) is indicated above each bar graph.

FIG. 8A-FIG. 8C: NOD-DR3 mice (n=4) were immunized with the 3-peptide cocktail (Tg.1571+TPO.758+GAD.492) on days 0 and 7. Mice were treated IV with S53 (cepharanthine) on days −2, −1, 5, and 6 at 1 mg/kg. Mice were sacrificed on day 21 and their splenocytes were stimulated with the peptides used for immunization (Tg.1571 (FIG. 8A), TPO.758 (FIG. 8B), GAD.492 (FIG. 8C)). Lymphocyte proliferation was determined by staining with carboxyfluorescein succinimidyl ester (CFSE). The Y-axis shows fold increased stimulation vs. control lymphocytes not stimulated with peptides. S53 (cepharanthine) significantly suppressed T-cell activation in treated mice when compared to vehicle treated controls.

### DETAILED DESCRIPTION

This disclosure provides methods of treating and/or preventing type 1 diabetes and/or autoimmune polyglandular syndrome type 3 variant in a subject.

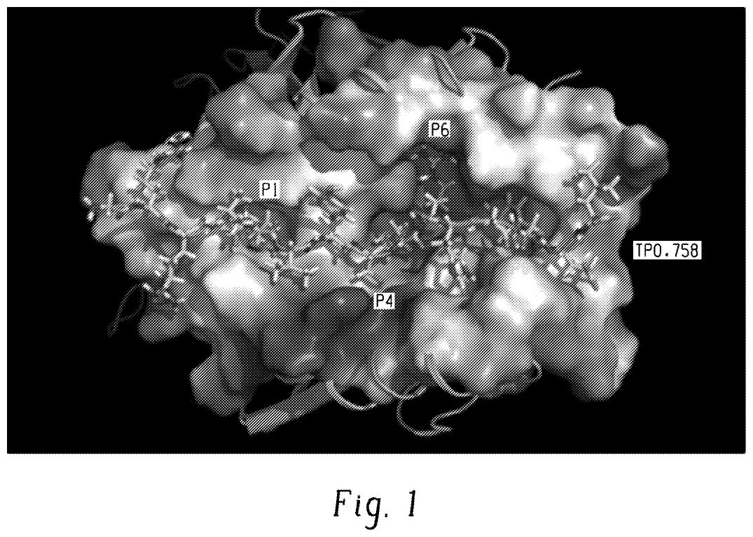

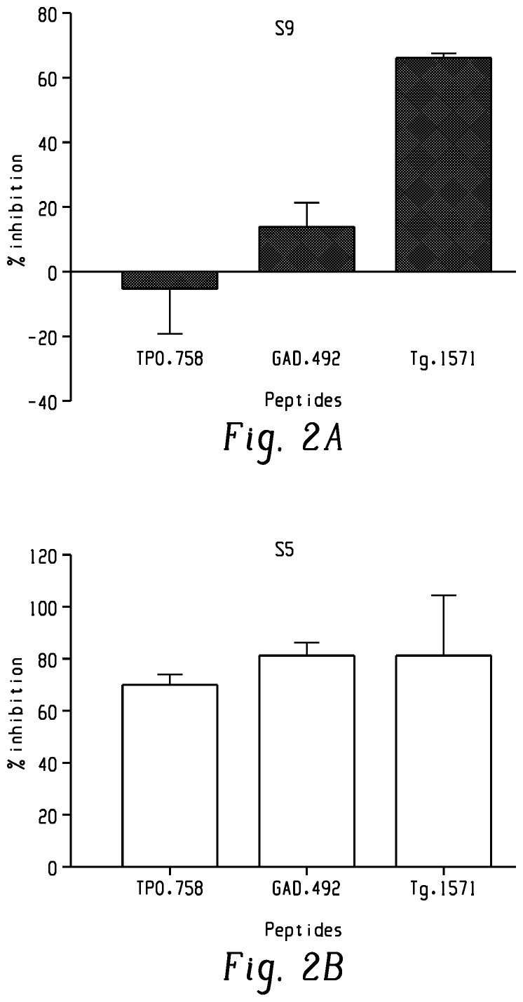

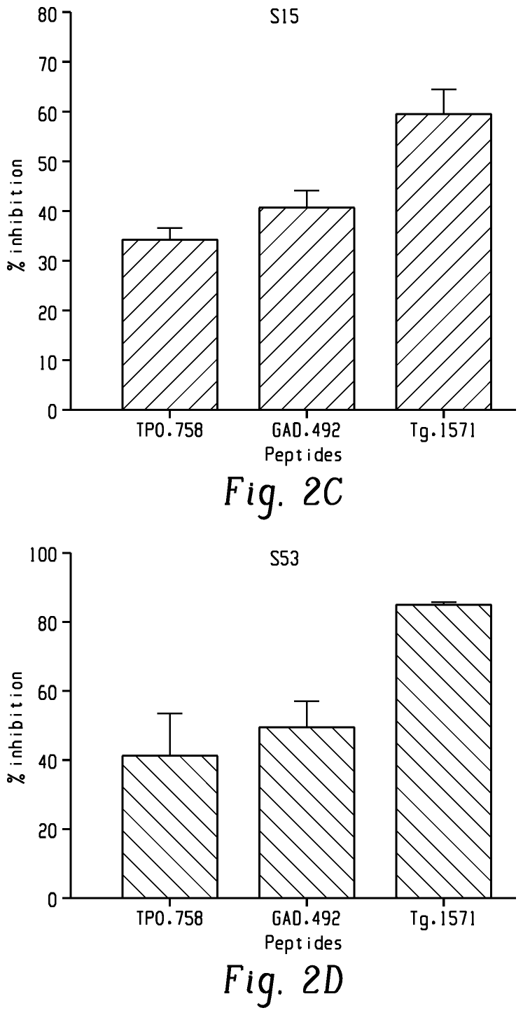

The co-occurrence of type 1 diabetes (T1D) and autoimmune thyroid disease (AITD) within the same individual (which happens in 20-30% of T1D patients) is commonly designated Autoimmune Polyglandular Syndrome type 3 variant (APS3v). HLA class II is the major susceptibility gene for T1D alone and when it develops together with AITD, resulting in APS3v. A unique amino acid signature in the HLA-DR3 pocket that predisposes individuals to APS3v has been previously identified by inventors. This unique HLA-DR3 pocket has been shown to be flexible and capable of binding and presenting both islet and thyroid peptides to T cells, thereby triggering T1D and AITD (APS3v). Three peptides, the thyroglobulin (Tg) peptide (Tg.1571), the glutamic acid decarboxylase 65 (GAD65) peptide (GAD.492), and thyroid peroxidase (TPO) peptide 758 (TPO.758), have been identified as the dominant peptides presented by HLA-DR3 and triggering the development of AITD and T1D (APS3v). An additional peptide (TPO.338) triggers only T-cell responses but not B-cell responses and therefore is not a dominant peptide (FIG. 2). FIG. 1 shows a representative structure of the interaction between human leucocyte antigen-DR3 (HLA-DR3) and thyroid peroxidase-758 (TPO.758). The sequences of the four APS3v peptides, as well as the peptide APO, used as a positive control peptide (since it is known to be a strong binder to HLA-DR3) are shown in Table 1.

TABLE 1
Sequence of the peptides used in ELISA screening
or immunizations.

| Peptide | Sequence |
|---|---|
| Tg.1571 | EKVPESKVIFDANAPVAVRSKVPDSEF |
| | (SEQ ID NO: 1) |
| GAD.492 | REGYEMVFDGKPQHTNVCF (SEQ ID NO: 2) |
| TPO.758 | ESGRRVLVYSCRHGYELQG (SEQ ID NO: 3) |
| APO | IPDNLFLKSDGRIKYTLNK (SEQ ID NO: 4) |
| TPO.338 | RQLRNWTSAEGLLRVHARL (SEQ ID NO: 5) |

As mentioned above, HLA-DR3 has been identified as the key HLA class II allele associated with T1D and AITD co-occurring in the same individual (i.e., APS3v). In addition, it has been demonstrated that HLA-DR3 is also the key HLA class II allele associated with a subset of T1D patients characterized by high levels and early development of glutamic acid decarboxylase (GAD)-binding antibodies (GADA). Together the T1D subsets—APS3v and T1D characterized by high GAD-binding antibody levels—may represent 40-50% of T1D patients, and in these subsets of T1D patients HLA-DR3 is the key HLA class II allele triggering the disease.

These findings illustrate that the HLA-DR3 binding pocket plays an important role in the development of two subsets of T1D. The present disclosure provides specific and unique inhibitors of the HLA-DR3 pocket as a precision medicine approach to treating or preventing T1D in those patients that carry the HLA-DR3 allele. Towards this goal, small molecules were screened for their ability to block the HLA-DR3 pocket, and a molecule, cepharanthine (S53), was found to be highly effective. In particular, it has been advantageously discovered that cepharanthine (S53) blocks HLA-DR3 and that the blocking of HLA-DR3 by cepharanthine suppresses autoreactive T-cell activation against islet and thyroid antigens. The results indicate that cepharanthine can thus be used (1) in the treatment and/or prevention of T1D patients that also have AITD (i.e., patients with APS3v) and carry the HLA-DR3 allele genotype and (2) in the treatment and/or prevention of T1D in patients having high GAD antibodies and carry the HLA-DR3 allele genotype.

Prior to describing the invention in detail, it is helpful to consider the following definitions.

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim.

All compounds are understood to include all possible isotopes of atoms occurring in the compounds. Isotopes include those atoms having the same atomic number but different mass numbers. The disclosure includes methods in which one or both of the compounds cepharanthine or a derivative thereof are isotopically enriched. For example, any of cepharanthine or a derivative thereof can be isotopically enriched with a non-radioactive isotope at one or more positions. By way of general example, and without limitation, isotopes of hydrogen include tritium and deuterium and isotopes of carbon include ¹¹C, ¹³C, and ¹⁴C.

The opened ended term “comprising” includes the intermediate and closed terms “consisting essentially of” and “consisting of.” Wherever an open ended aspect that may contain additional elements is contemplated (comprising language), more narrow aspects that contain only the listed items (consisting of language) are also contemplated.

“Pharmaceutical composition” means a composition comprising at least one active agent, such as cepharanthine, or a pharmaceutically acceptable salt, derivative, or solvate thereof, and at least one other substance, such as an excipient. An excipient can be a carrier, filler, diluent, bulking agent or other inactive or inert ingredients. Pharmaceutical compositions optionally contain one or more additional active agents. Pharmaceutical compositions meet the U.S. FDA's GMP (good manufacturing practice) standards for human or non-human drugs.

“Pharmaceutically-acceptable carrier” refers to a diluent, adjuvant, excipient, or carrier, other ingredient, or combination of ingredients that alone or together provide a carrier or vehicle with which a compound or compounds of the invention is formulated and/or administered, and in which every ingredient or the carrier as a whole is pharmaceutically acceptable. The pharmaceutically-acceptable carrier includes a carrier that is acceptable for veterinary use as well as human pharmaceutical use. Also included are any and all solvents, dispersion media, coatings, antibacterial and antifungal agents, and isotonic and absorption delaying agents. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. A “pharmaceutically acceptable carrier” includes both one and more than one such carrier.

A “patient” or a “subject” means a human or non-human animal in need of medical treatment. Medical treatment can include treatment of an existing condition, such as a disease or disorder or diagnostic treatment. In an aspect, the patient or the subject is a human patient or human subject. In an aspect the patient or subject is a domesticated companion animal such as a dog or cat.

“Providing” means giving, administering, selling, distributing, transferring (for profit or not), manufacturing, compounding, or dispensing.

“Administering” means giving, providing, applying, or dispensing by any suitable route. Administration of a combination of active agents includes administration of the combination in a single formulation or unit dosage form, administration of the individual active agents of the combination concurrently but separately, or administration of the individual active agents of the combination sequentially by any suitable route. The dosage of the individual active agents of the combination may require more frequent administration of one of the active agent(s) as compared to the other active agent(s) in the combination.

Therefore, to permit appropriate dosing, packaged pharmaceutical products may contain one or more dosage forms that contain the combination of active agents, and one or more dosage forms that contain one of the combination of active agents, but not the other active agent(s) of the combination.

“Treatment” or “treating” means providing the active agent (compound) disclosed herein as either the only active agent or together with at least one additional active agent sufficient to measurably reduce a type 1 diabetes (T1D) symptom or an autoimmune polyglandular syndrome type 3 variant (APS3v) symptom, slow progression of T1D or APS3v, or minimize the risk of developing T1D or APS3v. Treatment of the T1D or APS3v may be initiated before the subject presents symptoms of the disease.

“Prevention” or “preventing” as used herein includes (1)avoid the development of a disease in a subject at risk for the disease or (2) effecting a significant delay in the onset of symptomatic disease in subject at risk of developing symptomatic disease beyond the time when subject is predicted to develop symptomatic disease if untreated. A method of prevention usually starts before the obvious sickness of the disease. In T1D, which is the result of autoimmune destruction of the islet beta-cells and subsequent insulin deficiency and hyperglycemia, blocking the autoimmunity may be effective in preventing the development of T1D.

An “effective amount” or “therapeutically effective amount” of an active agent or a composition including the active agent means an amount effective, when administered to a subject, to provide a therapeutic benefit. The therapeutic benefit can include an amelioration of symptoms, a decrease in disease progression, or inhibiting the development of the disease. An effective amount can vary depending upon a variety of factors including the age, body weight, general health, sex, diet, time of administration, route of administration, rate of excretion, drug combination, and the severity of the particular disorder for the patient undergoing therapy.

A significant change is any detectable change that is statistically significant in a standard parametric test of statistical significance such as Student's T-test, where p<0.05.

“Derivative” of cepharanthine means any chemical modification to the structure of cepharanthine resulting in a stable alkaloid capable of interacting with the HLA-DR3 pocket with an affinity similar to that of cepharanthine. Examples of cepharanthine derivatives include bisbenzylisoquinoline (BBIQ) cyclic alkaloids that bear two coclaurine units joined head-to-tail or head-to-head such as fangchinoline, tetandrine, hernandezine, berbamine, daphnoline, or cycleanine.

The term “combination therapy” refers to the administration of two or more therapeutic (active) agents to treat a therapeutic condition or disorder described in the present disclosure. Such administration encompasses co-administration of these therapeutic agents in a substantially simultaneous manner, such as in a single dosage form having a fixed ratio of active ingredients or in separate dosage forms for each active ingredient. In addition, such administration also encompasses administration of each therapeutic agent in a sequential manner, either at approximately the same time or at different times. In either case, the treatment regimen will provide the beneficial effects of each therapeutic agent in the drug combination in treating the conditions or disorders described herein.

“Type I diabetes” or “T1D” is a chronic condition in which the pancreas of an affected subject produces little or no insulin.

“Autoimmune thyroid disease” or “AITD” occurs when the immune system of subject attacks or overstimulates the thyroid gland resulting in an overactive thyroid (hyperthyroidism) or an underactive thyroid (hypothyroidism) in the subject. Common forms of an autoimmune thyroid disorder include chronic lymphocytic thyroiditis, or Hashimoto's Thyroiditis (hypothyroidism), and Graves' disease (hyperthyroidism). Sometimes, AITD manifest only by the production of thyroid autoantibodies (e.g., thyroid peroxidase antibodies or thyroglobulin antibodies) without the development of hyperthyroidism or hypothyroidism.

“Autoimmune polyglandular syndrome type 3 variant” or “APS3v” as used herein refers to a syndrome in which both T1D and autoimmune thyroid disease develop in the same subject. APS3v is a subset of APS3 syndrome.

Cepharanthine (S53) (CAS Reg. No. 481-49-2) is a bisbenzylisoquinoline plant alkaloid produced by the plant Stephania cepharantha Hayata, and has the chemical formula:

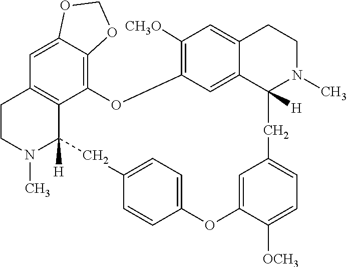

Cepharanthine is used in Japan for the treatment of different conditions, for example, chemotherapy-induced leukopenia, and has a high safety profile based on the Japanese experience. Cepharanthine or a pharmaceutically acceptable salt, solvate, or derivative thereof can be used in the methods disclosed herein. In an aspect, the solvate is a hydrate. In an aspect, cepharanthine derivatives are bisbenzylisoquinoline (BBIQ) cyclic alkaloids that bear two coclaurine units joined head-to-tail or head-to-head such as fangchinoline, tetandrine, hernandezine, berbamine, daphnoline, or cycleanine:

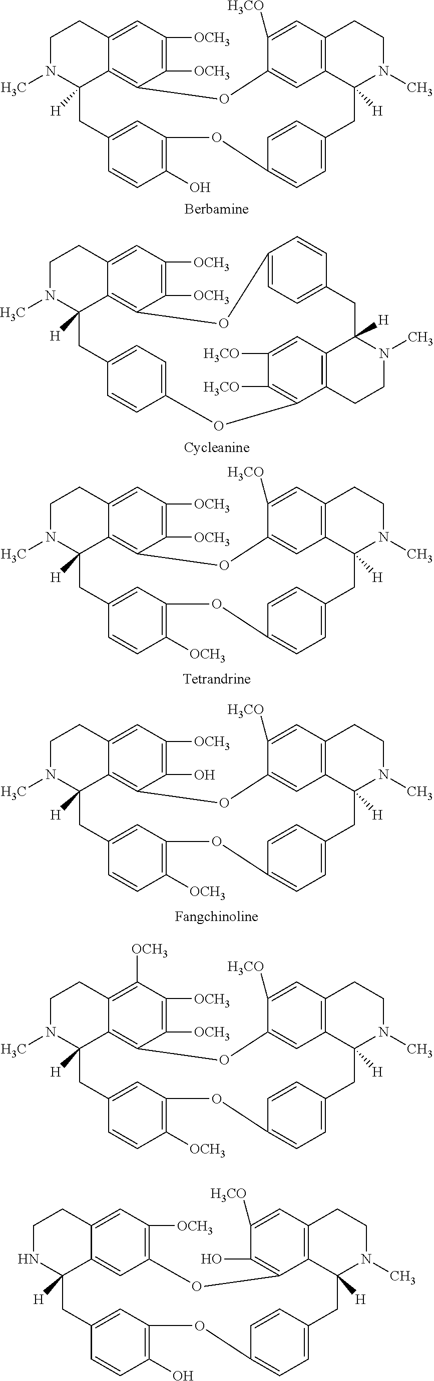

A pharmaceutically acceptable salt of cepharanthine includes salts that retain the biological effectiveness and properties of the compound, and which are not biologically or otherwise undesirable, and include derivatives of the disclosed compounds in which the parent compound is modified by making inorganic and organic, non-toxic, acid or base addition salts thereof. The salts can be synthesized from the parent compound by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable.

Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the cepharanthine parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

A salt of cepharanthine further includes solvates of the compound and of the compound salts. A “solvate” means cepharanthine or its pharmaceutically acceptable salt, wherein molecules of a suitable solvent are incorporated in the crystal lattice. An exemplary solvent is physiologically tolerable at the dosage administered. Exemplary solvents are ethanol, water, and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. In an aspect, the solvate is a hydrate.

The present disclosure provides safe, effective, specific, and unique inhibitors of the HLA-DR3 antigen binding pocket (“binding pocket”) as a precision medicine approach to treating or preventing T1D or APS3v in the subset of patients that carry the HLA-DR3 allele. In this regard, after screening small molecules that can block the HLA-DR3 pocket, it has been advantageously discovered that cepharanthine (S53) is highly effective at blocking the HLA-DR3 pocket.

The present disclosure provides a method of treating and/or preventing type 1 diabetes (T1D) in a subject including administering a therapeutically effective amount of cepharanthine or a pharmaceutically acceptable salt, solvate, or derivative thereof to the subject, wherein the subject has a human leucocyte antigen class II, DR3 allele (HLA-DR3) genotype. The methods described herein can reverse or prevent the autoimmune attack on beta cells in T1D patients by blocking HLA-DR3 which is needed to activate T-cells to attack the beta cells. Advantageously, because only DR3 is targeted while the other HLA class II molecules are not, the patients will not develop global immune suppression and should maintain a normal immune responses to infections. Also, advantageously, patients who have more advanced disease and have lost some of their beta cell function may also benefit from the methods described herein. In an aspect, the methods described herein can reduce the dose of insulin needed by the subject or completely eliminate the need for insulin. The methods can reduce the symptoms of type 1 diabetes and its complications such as elevated blood sugar level, elevated hemoglobin A1c level, nephropathy, neuropathy, retinopathy, ketoacidosis, glycosuria, hypoglycemic attacks, and combinations thereof.

In an aspect, the method of treating and/or preventing type 1 diabetes (T1D) in a subject further includes screening the subject for the HLA-DR3 genotype, for example, prior to administering the cepharanthine. In an aspect, the method includes administering a therapeutically effective amount of cepharanthine to the subject having the HLA-DR3 genotype.

In the methods disclosed herein, screening the subject for the HLA-DR3 allele genotype includes obtaining a blood sample from the subject and testing the blood sample to determine the HLA class II alleles of the subject. Such testing is also known as HLA typing. Methods of HLA typing to determine an HLA-DR3 genotype are known to those of skill in the art and are not limited herein. For example, a molecular method (e.g., polymerase chain reaction (PCR)-based) or an HLA-specific antibody based method can be used. Quest has commercial assay called SSO (Sequence Specific Oligonucleotides) which can be used in which they design oligonucleotides (primers) for PCR that are specific for the HLA-DR allele to be tested (for example HLA-DR3). This specific allele is used in a PCR reaction. An assay that is based on RFLP (Restriction Fragment Length Polymorphisms) which is well-known in the art may also be employed.

The disclosure further includes a method for use of cepharanthine in the treatment of two subsets of T1D subjects having the HLA-DR3 genotype: (1) a subject having T1D and autoimmune thyroid disease (AITD), i.e., a subject having APS3v; and (2) a subject having T1D and a high level of glutamic acid decarboxylase (GAD) antibody. The GAD antibody can be serum GAD antibody.

In an aspect, the disclosure provides a method of treating and/or preventing T1D in a subject having the HLA-DR3 genotype, and having T1D and autoimmune thyroid disease (AITD). In an aspect, the subject has autoimmune polyglandular syndrome type 3 variant (APS3v).

In an aspect, the disclosure provides a method of treating and/or preventing T1D in a subject having the HLA-DR3 genotype and having a high level of serum glutamic acid decarboxylase (GAD) antibody. GAD is an enzyme essential in the formation of gamma aminobutyric acid (GABA), which is an inhibitory neurotransmitter found in the brain.

Antibody which binds to GAD is referred to herein simply as GAD antibody or GAD autoantibody. The level of GAD antibody present in the serum of the subject can be tested using methods known to those of skill in the art. Such methods include, for example, enzyme linked immunosorbent assay (ELISA), surface plasmon resonance (e.g., Biacore™) immunoprecipitation, and immunoblotting (e.g., Western blotting). The level of GAD antibody is considered “high” when the amount of GAD antibody is greater than an amount of insulin antibody when both are measured as fold increase compared to the upper limit of normal levels, such as 2, 5, 10, 20, 30, 40, 50, 100-fold higher. A normal GAD antibody test in most ELISA assays is typically under 5 units/ml.

In the methods disclosed herein, the subject may be a human subject.

The present disclosure also provides a method of treating and/or preventing autoimmune polyglandular syndrome type 3 variant (APS3v) in a subject including administering a therapeutically effective amount of cepharanthine or pharmaceutically acceptable salt, solvate, or derivative thereof to the subject, wherein the subject has a human leucocyte antigen class II, DR3 allele (HLA-DR3) genotype.

In an aspect, the method of treating and/or preventing APS3v in the subject further includes screening the subject for the HLA-DR3 genotype.

In an aspect, the subject for the method of treating and/or preventing APS3v is a human subject.

In an aspect, the subject for the method of treating and/or preventing APS3v has a high level of serum glutamate decarboxylase (GAD) antibody.

Disclosed herein also is a method of treating and/or preventing type 1 diabetes (T1D) in a subject having T1D or at a high risk for developing T1D including determining that the subject has a human leucocyte antigen class II, DR3 allele (HLA-DR3) genotype; and administering a therapeutically effective amount of cepharanthine, or a pharmaceutically acceptable salt, solvate, or derivative thereof to the subject.

In an aspect, the determining step includes testing a sample from the subject to determine the HLA genotype of the subject. As discussed above, methods of HLA testing are known in the art, and are not limited herein.

In an aspect, the method further includes testing a serum sample from the subject for the presence of a high level of serum glutamate decarboxylase (GAD) antibody. As discussed above, methods for the detection of serum GAD antibody are known, and are not limited herein.

In an aspect, the method further includes determining that the subject comprises high serum GAD autoantibody levels.

In an aspect, the subject at risk for developing T1D is a subject having the HLA-DR3 genotype.

Cepharanthine or a pharmaceutically acceptable salt, solvate, or derivative thereof can be administered in the form of a composition including the compound and a pharmaceutically acceptable carrier. The composition can be administered to a subject using any known route of administration. For example, the administration can be systemic or localized to a specific site. Routes of administration include, but are not limited to, oral, topical, parenteral, intravenous, cutaneous, subcutaneous, intramuscular, inhalation or spray, sublingual, transdermal, intravenous, intrathecal, buccal, nasal, vaginal, rectal, or a combination thereof. In an aspect, the administration of cepharanthine or pharmaceutically acceptable salt, solvate or derivative thereof is oral or parenteral.

The cepharanthine or pharmaceutically acceptable salt, solvate or derivative thereof, or a composition including the cepharanthine or pharmaceutically acceptable salt, solvate or derivative thereof is formulated for administration to the subject in a suitable dosage form. The dosage form can be, for example, a capsule, a tablet, an implant, a troche, a lozenge, a minitablet, a suspension, an emulsion, a solution, an aerosol, an injectable, an ovule, a gel, a wafer, a chewable tablet, a powder, a granule, a film, a sprinkle, a pellet, a topical formulation, a patch, a bead, a pill, a powder, a triturate, a smart pill, a smart capsule, a platelet, a strip, or a combination thereof.

The methods of treatment disclosed herein include providing an effective amount of the cepharanthine or a pharmaceutically acceptable salt, solvate, or derivative thereof to a subject. The effective amount can be provided as a dosage form. The effective amount of cepharanthine or a pharmaceutically acceptable salt, solvate, or derivative thereof can be about 0.01 milligrams (mg) to about 10 mg per kilogram of body weight per day. In an aspect 0.1 mg to 500 mg, 1 mg to 200 mg, 1 mg to 100 mg, 1 mg to 500 mg, 1 mg to 200 mg, 1 mg to 100 mg, 1 mg to 50 mg, 10 mg to 500 mg, 10 mg to 200 mg, 10 mg to 100 mg, 10 mg to 500 mg 10 mg to 300 mg, 10 mg to 200 mg, 10 mg to 100 mg, 50 mg to 500 mg, 50 mg to 200 mg, 50 mg to 100 mg, 50 mg to 500 mg, 50 mg to 200 mg, of cepharanthine or a pharmaceutically acceptable salt, solvate, or derivative thereof, are administered daily to a patient. In an aspect, a dosage form including the cepharanthine or a pharmaceutically acceptable salt, solvate, or derivative thereof is provided to the patient. In an aspect the effective amount of cepharanthine, or a pharmaceutically acceptable salt, solvate, or derivative thereof is administered to the patient as a single dose or a plurality of doses. For example, the subject can be administered 1 to 4 daily doses. In an aspect the dosage of cepharanthine or a pharmaceutically acceptable salt, solvate, or derivative thereof is 100 to 500 mg, 300 mg, or 150 mg administered as 1 to 2 daily doses. In an aspect, a dosage form including the cepharanthine or a pharmaceutically acceptable salt, solvate, or derivative thereof is provided to the patient to lower an A1C level below 5.7%, and/or to maintain the A1C level below 5.7%. In an aspect, a dosage form including the cepharanthine or a pharmaceutically acceptable salt, solvate, or derivative thereof is provided to the patient to lower a blood sugar level for random blood sugar test below about 200 milligrams per deciliter (mg/dL) or about 11.1 millimoles per liter (mmol/L), for fasting blood sugar test below 100 mg/dL (5.6 mmol/L), and for the oral glucose tolerance test below 140 mg/dL (7.8 mmol/L).

Frequency of dosage may also vary depending upon the particular disease to be treated. However, for treatment of type 1 diabetes (T1D), a dosage regimen of 4 times daily or less is preferred, and a dosage regimen of 1 or 2 times daily is particularly preferred. Treatment regimens may also include administering the first active compound (cepharanthine or a derivative thereof) to the patient for a number of consecutive days, for example for at least 5, 7, 10, 15, 20, 25, 30, 40, 50, or 60 consecutive days. In certain aspects the first active compound is administered for a period of 1 to 10 weeks and the amount and frequency of dosage is such that concentration of the compound in the patient's plasma in never less than 50% of the patient's plasma Cₘₐₓ.

Treatment regimens may also include administering the first active compound to the patient for a number of days prior to pancreas transplant surgery (surgery to place a healthy pancreas from a deceased donor into a person whose pancreas no longer functions properly). For example, the first active compound may be administered to the patient for a number of consecutive days at 1 to 4 months prior to surgery.

Cepharanthine or a pharmaceutically acceptable salt, solvate, or derivative thereof, may be used alone or in combination with an additional active agent (therapeutic agent). Combination use includes an administering of the cepharanthine or a pharmaceutically acceptable salt, solvate, or derivative thereof and additional active agent in a single dosage form, or in separate dosage forms, either simultaneously or sequentially.

Doses of cepharanthine a pharmaceutically acceptable salt, solvate, or derivative thereof when used in combination with a second active agent are generally as described above. Doses and methods of administration of other therapeutic agents can be found, for example, in the manufacturer's instructions in the Physician's Desk Reference.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy

The cepharanthine or a pharmaceutically acceptable salt, solvate, or derivative thereof may be administered in combination with another active agent used for the treatment of type 1 diabetes (T1D), autoimmune thyroid disease (AITD), or Autoimmune Polyglandular Syndrome type 3 variant (APS3v). The additional active agent used for the treatment of T1D can include insulin. Any form of insulin suitable for the treatment of T1D can be used including, for example, insulin aspart (NovoLog™, FlexPen™, Fiasp™), insulin glulisine (Apidra™), insulin lispro (Humalog™); an intermediate-acting insulin, for example, insulin isophane (Humulin N™, Novolin N™); a long-acting insulin, for example, insulin degludec (Tresiba™), insulin detemir (Levemir™), insulin glargine (Lantus™), insulin glargine (Toujeo™); a combination insulin, for example, NovoLog™ Mix 70/30 (insulin aspart protamine-insulin aspart), Humalog™ Mix 75/25 (insulin lispro protamine-insulin lispro), Humalog™ Mix 50/50 (insulin lispro protamine-insulin lispro), Humulin™ 70/30 (human insulin NPH-human insulin regular), Novolin™ 70/30 (human insulin NPH-human insulin regular), Ryzodeg™ (insulin degludec-insulin aspart). The insulin can be administered through any suitable means, for example, by injection or through the use of an insulin pump or artificial pancreas. Other active agents used for the treatment of T1D include an amylinomimetic drug such as pramlintide (SymlinPen™ 120, SymlinPen™ 60), metformin, teplizumab, anti-thymocyte globulin, or a combination thereof.

Active agents for the treatment of AITD include levothyroxine (Synthroid™, Tirosint™, Levoxyl™, Levothroid™, Unithroid™), beta blockers such as propranolol and metoprolol, methimazole (Tapazole™), propylthiouracil (PTU]), Carbimazole, radioactive iodine, or a combination thereof.

The additional active agent can also include those used for the treatment of type II diabetes. Such active agents include, for example, metformin (Fortamet™, Glumetza™); sulfonylureas such as glyburide (DiaBeta™, Glynase™), glipizide (Glucotrol) and glimepiride (Amaryl™); glinides such as repaglinide and nateglinide; thiazolidinediones such as rosiglitazone (Avandia™) and pioglitazone (Actos™); DPP-4 inhibitors such as sitagliptin (Januvia™), saxagliptin (Onglyza™) and linagliptin (Tradjenta™); GLP-1 receptor agonists such as exenatide (Byetta™, Bydureon™), liraglutide (Saxenda™ Victoza™) and semaglutide (Rybelsus™, Ozempic™); SGLT2 inhibitors such as canagliflozin (Invokana™), dapagliflozin (Farxiga™) and empagliflozin (Jardiance™); aspirin; or a combination thereof.

In an aspect, the methods of treating and/or preventing disclosed herein further include administering an effective amount of an additional active agent to the subject, wherein the additional active agent includes insulin, an amylinomimetic drug, or a combination thereof. In an aspect, the additional active agent includes a short-acting insulin, a rapid-acting insulin, an intermediate-acting insulin, a long-acting insulin, a combination insulin, pramlintide, or a combination thereof.

In an aspect, the method of treating and/or preventing autoimmune polyglandular syndrome type 3 variant (APS3v) in a subject further includes administering an effective amount of an additional active agent to the subject, wherein the additional active agent comprises insulin, an amylinomimetic drug, or a combination thereof. In an aspect, the additional active agent includes a short-acting insulin, a rapid-acting insulin, an intermediate-acting insulin, a long-acting insulin, a combination insulin, pramlintide, or a combination thereof.

The methods disclosed herein are also useful for treatment of mammals other than humans, including for veterinary applications such as to treat horses and livestock, e.g., cattle, sheep, cows, goats, swine and the like, and pets (companion animals) such as dogs and cats.

### Materials and Methods

Peptide Synthesis: Peptides were custom-synthesized by Genscript (Piscataway, NJ). The following peptides were used in the study: (1) the thyroglobulin (Tg) peptide Tg.1571; (2) the glutamic acid decarboxylase 65 (GAD65) peptide GAD.492; (3) and the thyroid peroxidase (TPO) peptide TPO.758. The apopeptide (APO) that was previously shown to be the best binder to the HLA-DR3 pocket was used in the initial small molecule screening.

Materials: Cepharanthine (S53) used in these studies was purchased as a beige powder from Microsource Discovery Systems (Gaylordsville, CT). The identity and purity of the sample was verified. Other small molecules used in this study were purchased from ChemBridge (San Diego, CA).

Dose Response of Cepharanthine Blocking Peptide Binding to APS3v-HLA-DR3: 0.012 mg/ml of recombinant APS3v-HLA-DR3 was incubated with 10 pM biotinylated peptides [APO, Tg.1571, GAD.492, or TPO.758 (Genscript)], together with decreasing doses of cepharanthine (0.4 mM, 0.2 mM, 0.1 mM, 0.05 mM), for 48 h at 37° C. in a binding buffer. An in vitro binding assay was performed. Briefly, 0.012 mg/ml of recombinant APS3v-HLA-DR3 was incubated with 10 pM biotinylated peptides [APO, Tg.1571, GAD.492, or TPO.758 (Genscript) together with 0.4 mM of the small molecules, for 48 h at 37° C. in a binding buffer [0.1% BSA/PBS with 0.05% Triton™ (PBST), Sigma-Aldrich]. On the day before the immunoassay was performed, a 96-well DELFIA yellow plate (PerkinElmer Life Science) was coated overnight with 20 μg/ml of L243 antibody [Hybridoma was purchased from ATCC, catalogue number HB-55, and IgG was purified by QED Bioscience (San Diego, CA)] in bicarbonate buffer, pH 9.6 (Sigma-Aldrich). L243 is a monoclonal antibody that specifically recognizes the DRa chain of HLA-DR when it is properly folded and complexed with the b chain. The plate was then washed with DELFIA wash buffer (diluted 1:25 from DELFIA wash concentrate, PerkinElmer) to wash off the excess L243 antibody. Blocking was performed using 2.5% BSA in PBS at room temperature for 1 h. After washing 4 times, 100 μl of the pre-incubated mix (containing recombinant APS3v-HLA-DR3 protein, APO, and small molecules) were added onto the plate and shaken at slow speed for 2 h at room temperature. After washing 4 times, DELFIA Europium-labeled streptavidin (PerkinElmer) diluted in DELFIA assay buffer (PerkinElmer) was added for 30 min and shaken at slow speed at room temperature. After washing for 6 times, DELFIA Enhancement Solution was added for 1 h until the optimal signal was reached. Time-resolved fluorescence was measured using the BMG reader (BMG Labtech, Cary, NC).

Mice: Female humanized NOD-DR3 mice used in this study are knockout for murine MHC class II and express DRB1*0301 and confirmed by sequencing to contain the 4 critical amino acids of the APS3v-HLA-DR3 pocket.

Induction of T-Cell Response to Islet and Thyroid Autoantigens in Humanized NOD-DR3 Mice as a Model of APS3v: Previously we have identified 4 thyroid and islet peptides that triggered T-cell responses inNOD-DR3mice—Tg.1571,GAD.492, TPO.338 and TPO.758 (12). However, only 3 of these peptides (Tg.1571, GAD.492, and TPO.758) elicited significant B-cell (antibody) responses. Therefore, we concluded that these 3 peptides are the major T-cell epitopes in APS3v and used them to induce a model of APS3v in humanized NOD-DR3mice. To induce the APS3v model, 22 female NOD-DR3 mice 4-6 weeks old were co-immunized subcutaneously with these three peptides: Tg.1571, GAD.492 and TPO.758 (100 μg each peptide), in Complete Freund's Adjuvant (CFA, Sigma-Aldrich). Mice were immunized with peptides on day 0 and boosted on day 7, then sacrificed on day 21. As negative control, humanized NOD-DR3 littermates were immunized with PBS emulsified in CFA.

Lymphocytes Isolation: Spleen and draining lymph nodes were collected from mice upon sacrifice. Lymphocytes were isolated as described in the art. Briefly, the spleens and draining lymph nodes were harvested in complete RPMI (Corning, NY) supplemented with 10% FBS (Sigma-Aldrich) and 1 mM sodium pyruvate (Sigma-Aldrich). They were cut and pressed in circular motion using a plunger from a 10 ml syringe. The suspension was filtered through a 100 μm cell strainer twice and centrifuged 200×g for 10 min. The pellet was washed with RPMI and centrifuged one more time. 5 ml Ammonium-Chloride-Potassium (ACK) lysis buffer was added to remove erythrocytes from the spleen. After 5-min incubation with ACK lysis buffer at room temperature with occasional shaking, cells were centrifuged at 200×g for 10 min. The pellet was resuspended in RPMI and the cells were counted and plated.

Cytokine Assays: Milliplex mouse cytokines/chemokine magnetic panel (Catalog no. MCYTOMAG-70K, EMD Millipore Corporation, Billerica, MA) was used to assay the cytokines, as described in the art. Briefly, splenocytes were plated at 2×106 cells per well in 500 μl of medium (RPMI/10% FBS). Supernatants from stimulated lymphocytes were collected 48 h after stimulation with peptides and stored in −80° C. until the assay was performed. The 96-well plate supplied in the kit was washed with the wash buffer supplied and the plate was shaken for 10 min at room temperature. Standards and quality controls were added, followed by the samples. The pre-mixed beads (Interferon gamma and IL-2) were sonicated, vortexed and added to the wells. After shaking the plate overnight at 4° C., the plate was washed twice with wash buffer. Detection antibodies were added for 1 h at room temperature, and Streptavidin-Phycoerythrin was added for 30 min at room temperature. The plate was washed twice and sheath fluid was added to resuspend the beads for 5 min before reading in Luminex 200 with xPONENT software (Luminex, Austin, Texas).

Purification of Mouse Thyroglobulin (Tg): Mouse thyroglobulin was purified using a modified procedure. Briefly, mouse thyroid lysate in 10 mM HEPES pH 7.4, 100 mM NaCl buffer was loaded onto a 5 mL HiTrap® Q anion exchange column and was run at 4 ml/min, using a linear gradient from 0.1 to 1 M NaCl. Mouse Tg eluted in a peak spanning from 270 to 440 mM NaCl.

Autoantibody Measurements: Sera were collected from mice upon sacrifice and stored in −20° C. Nunc™ Maxisorp™ ELISA plate (Thermo Fisher Scientific, Waltham, MA) was coated with 10 μg/ml of either Tg.1571, GAD.492, TPO.758 or mouse Tg in bicarbonate buffer (Sigma-Aldrich) at pH 9.6 and incubated overnight. The ELISA plate was washed 4 times with PBS supplemented with 0.05% Tween (Sigma-Aldrich) [PBST]. After blocking with 2.5% BSA in PBST for 1 h at 37° C., the plate was washed with PBST for 6 times. 100 μl of the diluted sera (1:100 in 0.5% BSA/PBS) was added for 2 h with slow shaking at room temperature. After washing for 6 times with PBST, anti-mouse IgG secondary antibody (Sigma-Aldrich) was added at 1:30,000 dilution in 1% BSA/PBST and incubated for 30 min at 37° C. After washing the plate for 4 times with PBST, the freshly prepared paranitrophenylphosphate substrate (Sigma-Aldrich) was added for 1 h and the plate was read at 405 nm using the BMG reader (BMG Labtech, Cary, NC).

GAD antibody ELISA was performed using Glutamic Acid Decarboxylase (GAD) Autoantibody ELISA kit (Kronus, Star, ID). Briefly, standards, controls and serum samples were added to wells pre-coated with GAD and incubated for 1 h with shaking. After three washes using wash buffer supplied in the kit, GAD65-biotin was added for 1 h with shaking. Followed by another three washes, streptavidin-peroxidase was added for 20 min with shaking. After 3 more washes with wash buffer and one wash with deionized water, peroxidase substrate was added for 20 min, followed by stop solution. Absorbance at 450 nm was measured using the BMG reader (BMG Labtech, Cary, NC).

Free T4 Measurements: Sera were collected from mice and were assayed for free thyroxin (fT4) measurements using free thyroxine ELISA kit (Alpha Diagnostic International, San Antonio, TX, catalog no. 1110). Assay was performed according to manufacturer's protocol. The standards were run simultaneously with the serum samples to obtain a standard curve. Briefly, serum samples were added to the wells pre-coated with T4-specific antibodies. Diluted enzyme conjugate was added for 1 h at 37° C., followed by three washes using the wash buffer supplied in the kit. TMB substrate was added for 15 min at 37° C., followed by stop solution. Absorbance at 450 nm was measured using a BMG ELISA reader (BMG Labtech).

Small Molecule Inhibition of Cytokine Production Ex Vivo: Eight NOD-DR3 mice were immunized with the three thyroidal/islet peptides. Upon sacrifice, splenocytes were incubated with 1 μM of small molecules (S5, S9, S15, S53) together with TPO.758 (20 μg/ml), GAD.492 (20 μg/ml) or Tg.1571 (20 μg/ml) respectively to assess the blockade of these peptides' presentation by the tested small molecules. Supernatants from stimulated lymphocytes were collected 48 h after stimulation with peptides and small molecules, and stored in −80° C. until the cytokine assay was performed.

Blocking the Induction of APS3v by cepharanthine In Vivo: Four humanized NOD-DR3 mice were injected with 125 μg of cepharanthine intraperitoneally (IP) on days −2 and −1 prior to the immunization with the peptide mix of Tg.1571, GAD.492 and TPO.758 on day 0; mice were injected with cepharanthine IP again on days 5 and 6 prior to immunization with the same peptide mix on day 7. As controls, four humanized NOD-DR3 littermates were injected with the vehicle that was used to dissolve cepharanthine for in vivo studies [ethanol: PEG400 (Fisher Scientific): saline (5:20:75)] (Frontage Laboratories, Exton, PA) using the same timeline for immunizations.

T-Cell Stimulation and CFSE Analysis: Cells harvested from the spleen and draining lymph nodes of immunized mice were resuspended at 2×106 cells/ml in 0.1% BSA/PBS. 1×106 cells were labeled with 1.5 μM carboxyfluorescein diacetate succinimidyl ester (CFSE) (Life Technologies). After incubating for 10 min at 37° C., the staining was terminated by the addition of 4 volumes of ice-cold RPMI with 10% FBS. After incubating on ice for 5 min, the cells were washed three times with fresh RPMI and resuspended in fresh medium for counting.

The CFSE-labeled cells were plated at 2×105 cells/well in 100 μl medium (RPMI, 10% FBS). The cells were incubated with: medium, Tg.1571, GAD.492, TPO.758 (20 μg/ml), a negative control peptide (NC) (20 μg/ml), or mouse anti-CD3/CD28 beads (Life Technologies) that activate all CD4+ T-cells nonspecifically, as a positive control. The cells were collected after 5 days for flow cytometry analysis. All experiments were performed in quadruplicates. The results were analyzed using Flowjo (Tree Star, Ashland, OR). The stimulation index was calculated by using the following formula: stimulation index=[% proliferating lymphocytes (peptide/mitogen-treated)]/[% proliferating lymphocytes (medium-treated)].

Histology: Thyroid glands and pancreases were dissected and removed from immunized NOD-DR3 mice after sacrifice and stored in 10% formaldehyde until processing. After creating paraffin tissue blocks, thyroids and pancreases were sectioned and stained with hematoxylin-eosin for histological analysis (Histology and Comparative Pathology Core, Albert Einstein College of Medicine).

MD Simulations of the Complex of cepharanthine With APS3v-HLA-DR3: The docked structure of the cepharanthine in the APS3v-HLADR3 was used to construct the system for MD simulations. The system was placed in a truncated octahedral box filled with waters and ions with walls at a distance of 10 Å from the solute. The solute was positionally restrained and the system was minimized, heated and equilibrated by gradually relaxing the restraints. A production run at NPT was conducted for 1 ms. The trajectory was written to produce 50,000 structures. The stripped trajectory was clustered by 2D-rms to produce three clusters: the first was a transient structure for the first 6.5 ns and the other two represent two clusters in which cepharanthine undergoes a minor conformational change. These two clusters were distributed at a ratio of 3:1. We selected the cluster center of the larger cluster as a representative structure of the complex between cephranthine and the APS3v-HLA-DR3 (FIG. 1).

Statistical Analysis: Prism 5 software was used to perform the statistical analysis. Student's t test (unpaired t test, one-tailed) was used for comparisons of means of the experimental vs. control groups for each of the continuous variables measured. A p value <0.05 was considered statistically significant.

### EXAMPLES

### Example 1: Virtual Screening of Small Molecules Blocking APS3v-HLA-DR3

A virtual screen identified 100 small molecules that were predicted to block the unique APS3v-HLA-DR3 pocket. To produce a manageable selection of compounds for validation we clustered the 100 compounds by their simplified molecular input line entry system (SMILES) similarity into 20 clusters. Of the 20 clusters we selected the top scoring members in each cluster. We have enhanced this list to include a total of 11 compounds that blocked APS3v-HLA-DR3, the key HLA-DR3 pocket that triggers APS3v. We tested the selected 11 small molecules identified by the virtual screen in our in vitro ELISA, to test whether the 11 small molecules can block APO peptide (the strongest known peptide binder to HLA-DR3) binding to recombinant APS3v-HLA-DR3.

The virtual screen and ELISA confirmation identified 4 small molecules that showed significant (≥50%) inhibition of peptide binding to HLA-DR3: S5, S9, S15, and S53 (cepharanthine) (FIG. 2A-FIG. 2D). The structures of S5, S9, and S15 are shown below.

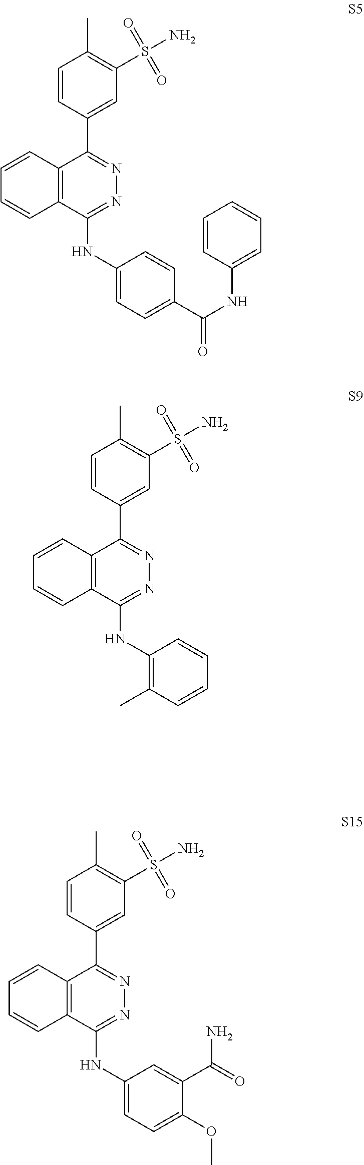 [SMILES: Cc1ccc(-c2nnc(Nc3ccc(C(=O)Nc4ccccc4)cc3)c3ccccc23)cc1S(N)(=O)=O ; Cc1ccccc1Nc1nnc(-c2ccc(C)c(S(N)(=O)=O)c2)c2ccccc12 ; COc1ccc(Nc2nnc(-c3ccc(C)c(S(N)(=O)=O)c3)c3ccccc23)cc1C(N)=O]

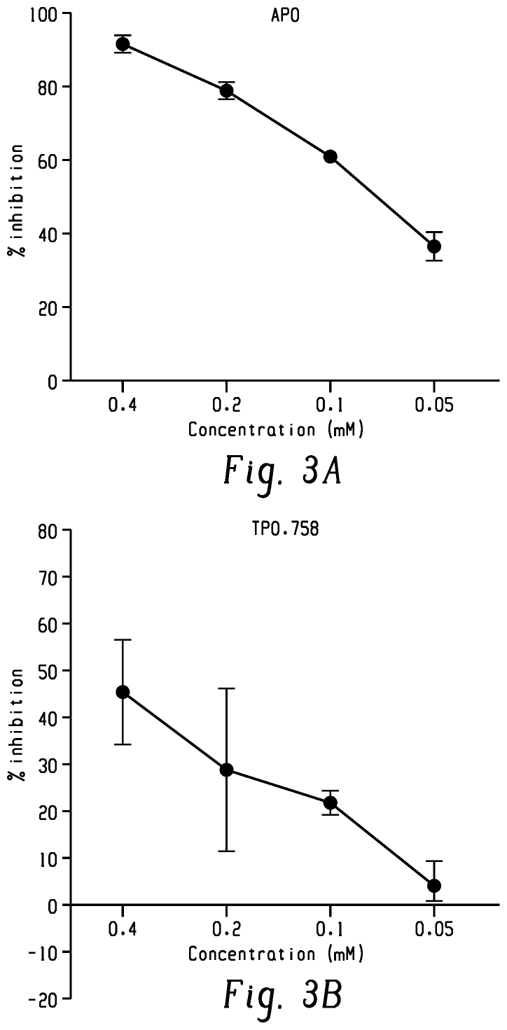

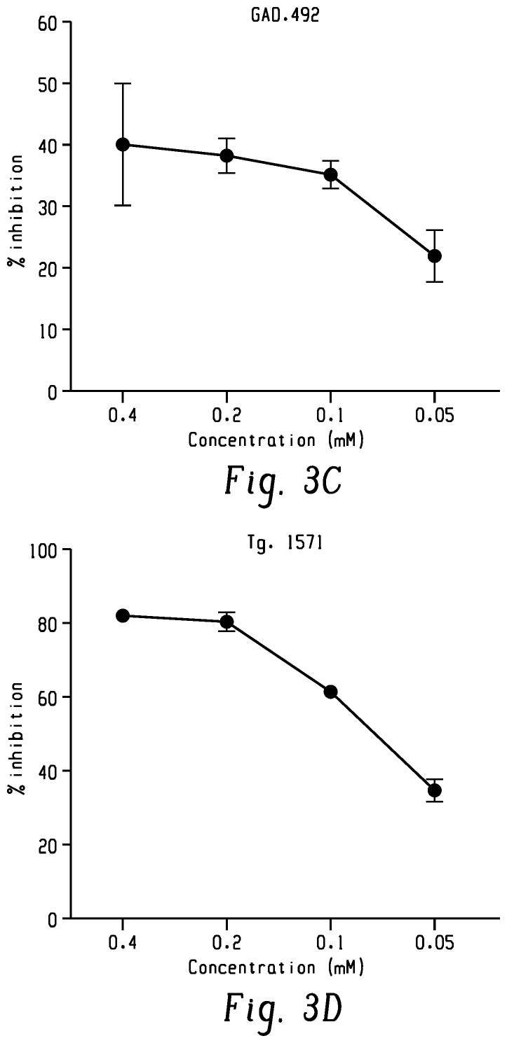

Of the 4 validated small molecules we selected S53 for further testing because it is the only one that has been used in humans before. Recombinant APS3v-HLA-DR3 was incubated with biotinylated APO, TPO.758, GAD.492 or Tg.1571, either alone or with decreasing doses (0.4 mM, 0.2 mM, 0.1 mM, 0.05 mM) of S53. ELISA was performed as previously described. S53 blocked the binding of each peptide in a dose-dependent manner (FIG. 3A-D).

### Example 2: Generating a Humanized Mouse Model of APS3v Showing Autoimmune Responses to Thyroid and Islet Major Autoantigens

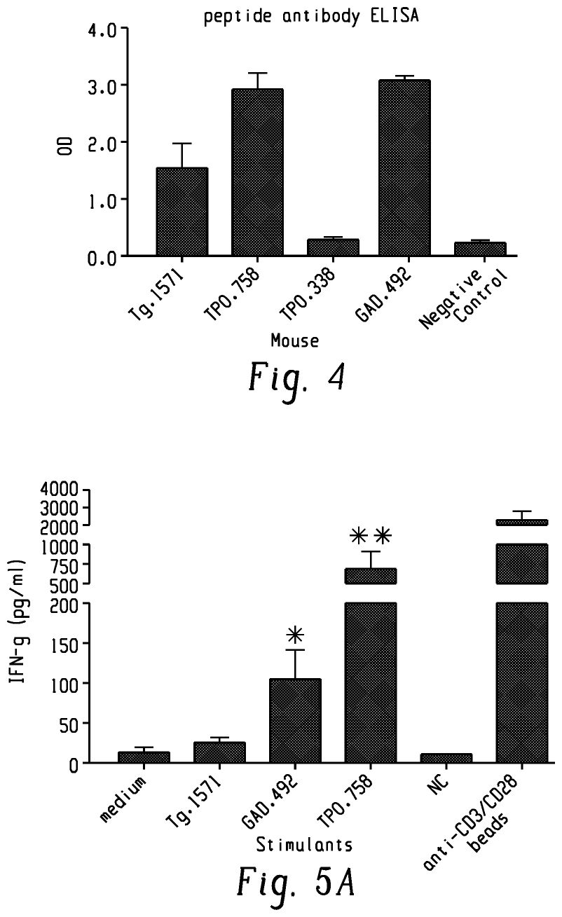

Four peptides presented by HLA-DR3, three thyroidal peptides (Tg.1571, TPO.338, and TPO.758) and one islet peptide (GAD.492), were identified as triggering the development of AITD and T1D (APS3v) and are considered to be major APS3v epitopes. This was confirmed by: (1) their ability to bind with high affinity to the VAVY B-cell line which is homozygous for HLA-DR3; and (2) their ability to elicit strong T-cell responses. However, only 3 peptides (Tg.1571, TPO.758, and GAD.492) elicited strong antibody responses in non-obese diabetic mice which carry and express the human HLA-DR3 gene (NOD-DR3 mice) associated with APS3v (FIG. 2). As shown in FIG. 4, NOD-DR3 mice (carrying the APS3v-DR pocket signature) immunized with the 4 peptides that elicited strong T-cell responses (data not shown), three of which (Tg.1571, TPO.758, GAD.492) also induced strong antibody responses in the immunized mice, illustrating that the 3 peptides that elicited both T-cell and antibody responses are the major peptides triggering APS3v.

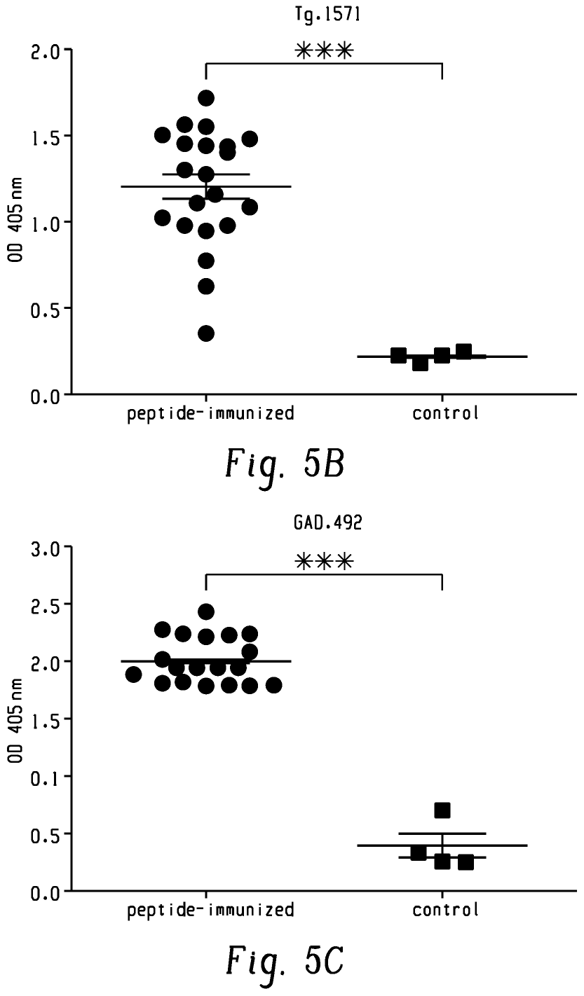

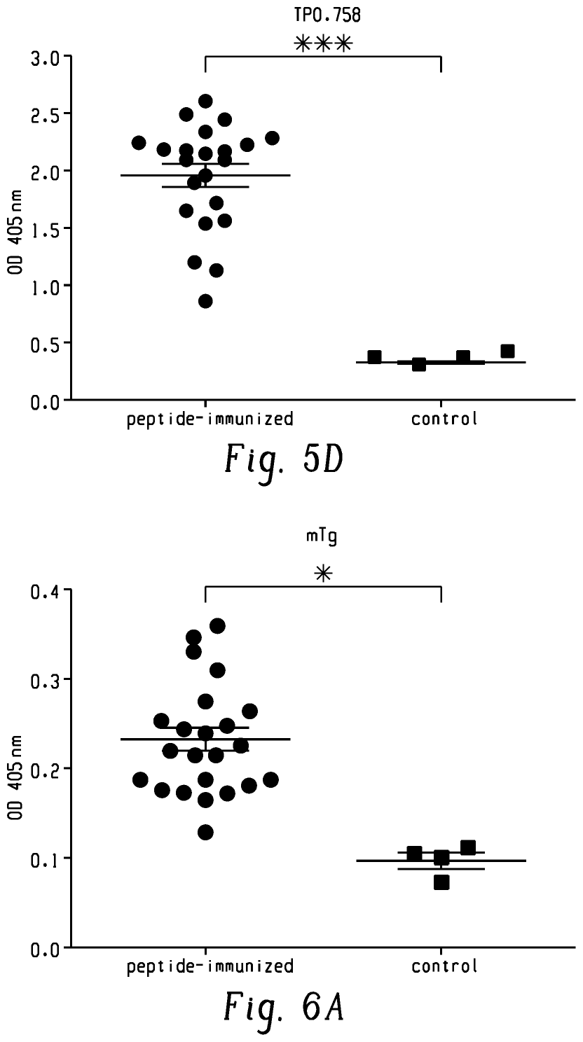

Twenty-two humanized NOD-DR3 mice were co-immunized with a peptide mix of Tg.1571, GAD.492 and TPO.758 together with adjuvant. Splenocytes and draining lymph node cells of immunized mice were isolated and stimulated with either: (1) Tg.1571, GAD.492, or TPO.758, thyroid and islet peptides that were previously shown to bind strongly to APS3v-HLA-DR3; (2) unrelated peptide (negative control); or (3)mouse anti-CD3/CD28 beads (positive control), for 48 hours to test for T-cell activation of cytokine responses using the Milliplex mouse cytokines/chemokine magnetic panel from EMD Millipore. Lymphocytes isolated from mice co-immunized with Tg.1571, GAD.492 and TPO.758 showed strong interferon gamma (IFN-g) responses to GAD.492 (99.6 μg/ml) (p<0.05), TPO.758 (716.62 μg/ml) (p<0.01), and Tg.1571 (24.2 μg/ml) (p=0.1511),compared to the control (FIG. 5A).

Sera from humanized NOD-DR3mice co-immunized with Tg.1571, GAD.492 and TPO.758 (n=22) were collected and stored in −20° C. until ELISA was performed for antibody measurements. Sera was added to ELISA plates coated with each peptide (Tg.1571, GAD.492 and TPO.758) respectively and incubated for 2 h before anti-mouse IgG secondary antibody and para-nitrophenylphosphate substrate were added for measurements using the BMG reader. Humanized NOD-DR3 mice coimmunized with Tg.1571, GAD.492 and TPO.758 developed a strong humoral antibody response to each of the peptides (p<0.001, compared to PBS-immunized controls) (FIGS. 5B-D). The immunized NOD-DR3 mice also developed high levels of antibodies against mouse Tg protein (p<0.05, compared to controls) (FIG. 4A). We also checked if the mice developed antibodies against GAD protein. Two out of 22 immunized mice developed antibodies against GAD protein while none of the controls developed GAD antibodies (data not shown).

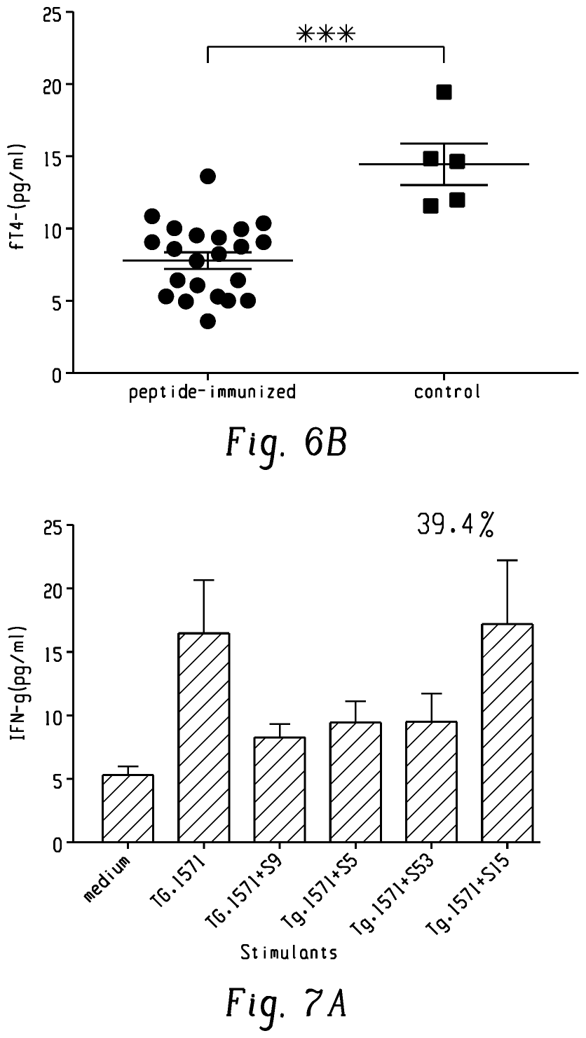

Twenty-two NOD-DR3 mice co-immunized with Tg.1571, GAD.492 and TPO.758 with adjuvant showed significantly lower free T4 levels (7.77 μg/ml) compared to control NODDR3 mice immunized with PBS and adjuvant (14.84 μg/ml) (p<0.001) (FIG. 6B).

### Example 3: Thyroid and Pancreatic Histology

Upon co-immunization of thyroid (Tg.1571, TPO.758) and islet (GAD.492) peptides, thyroid or islet lymphocytic infiltration was not observed in the 22 humanized NOD-DR3 mice (data not shown).

### Example 4: Cepharanthine (S53) Blocks T-Cell Activation by Thyroid and Islet Peptides Ex Vivo

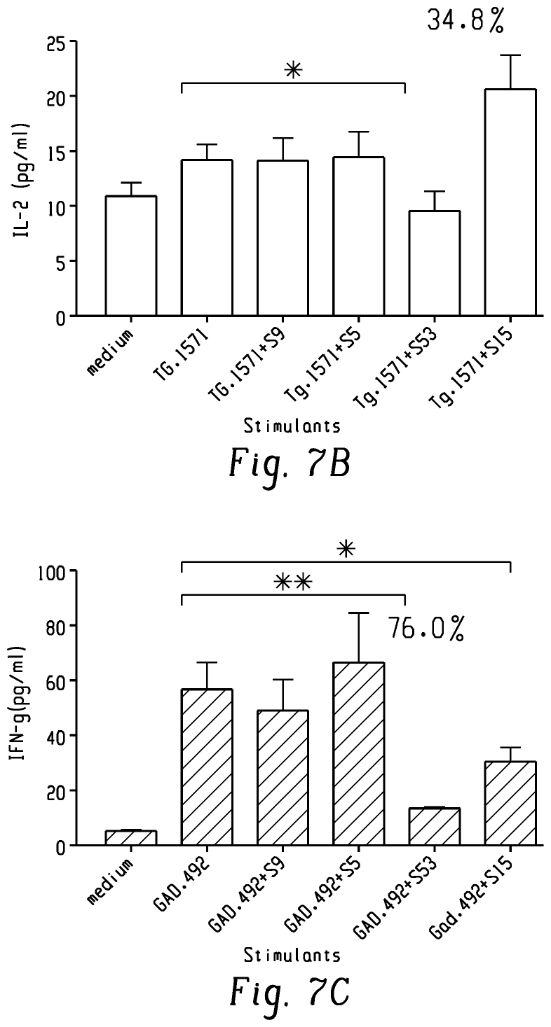

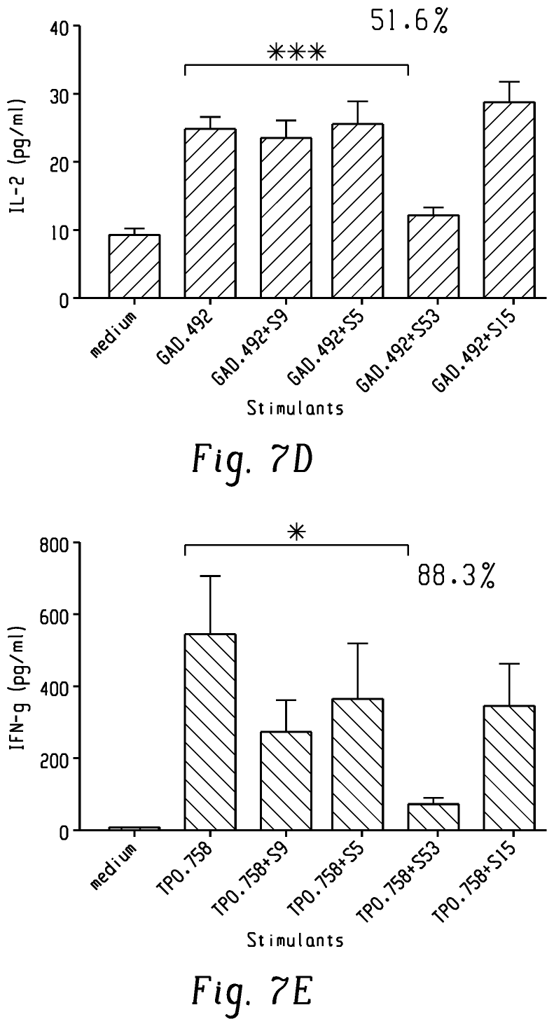

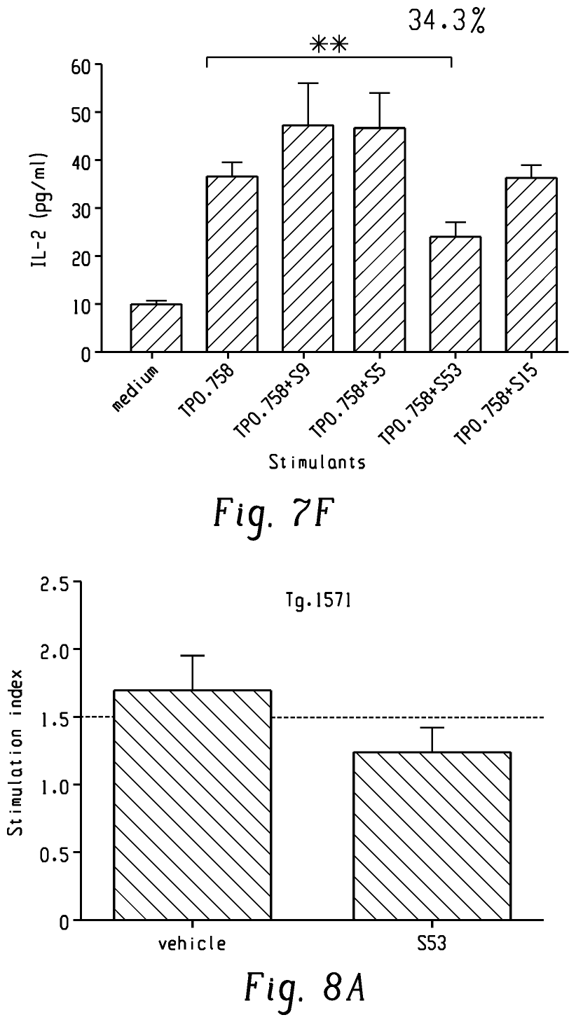

Upon sacrifice of the 8 NOD-DR3 mice co-immunized with Tg.1571, GAD.492 and TPO.758, their splenocytes and draining lymph node cells were harvested and stimulated with Tg.1571, GAD.492 or TPO.758, in the presence or absence of each of the 4 small molecule hits (S5, S9, S15 and S53) to assay for cytokine production using the Milliplex mouse cytokines/chemokine magnetic panel from EMD Millipore (see Materials and Methods for details). Both S15 and S53 significantly reduced GAD.492− induced IFN-g production [reduced from 56.3 μg/ml (GAD.492) to 30.1 μg/ml (GAD.492+S15, p<0.05) and 13.5 μg/ml (GAD.492+S53, p<0.01)]. Interestingly, only S53 reduced GAD.492-induced IL-2 production (reduced from 25.2 μg/ml to 12.3 μg/ml, p<0.001) (FIG. 7A,B). Only S53 significantly inhibited TPO.758-induced IFN-g (from 535.6 μg/ml to 62.3 μg/ml, p<0.05) and IL-2 (from 36.7 μg/ml to 24.1 μg/ml, p<0.01) production (FIG. 7C,D). Similarly, only S53 significantly inhibited Tg.1571-induced IL-2 production, from 12.4 μg/ml to 8.1 μg/ml (p<0.05) (FIG. 7E,F), albeit it did not significantly reduce IFN-g production in response to Tg.1571.

### Example 5: Cepharanthine (S53) Blocks Activation of T-Cells to Thyroid and Islet Peptides In Vivo

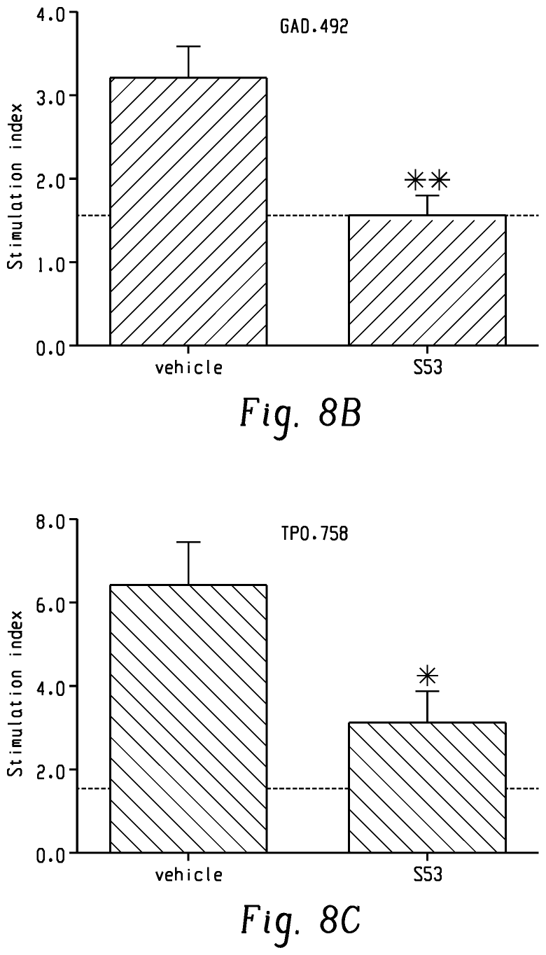

Four humanized NOD-DR3mice were treated with cepharanthine (S53) or vehicle (used to dissolve cepharanthine) for 2 consecutive days before each of the two immunizations with Tg.1571,GAD.492 and TPO.758 peptide mix. At sacrifice, splenocytes and draining lymph node cells were incubated with each peptide and labeled with CFSE for 5 days for T-cell stimulation analysis. Briefly, the CFSE labeled cells were plated at 2×105 cells/well in 100 μl medium (RPMI, 10% FBS).The cells were incubated with: medium, Tg.1571, GAD.492, TPO.758 (20 μg/ml), a negative control peptide (NC) (20 μg/ml), or mouse anti-CD3/CD28 beads (Life Technologies) that activate all CD4+ T-cells non-specifically, as a positive control. The cells were collected after 5 days for flow cytometry analysis (see Materials and Methods for details). S53 significantly suppressed T cell proliferation induced by GAD.492 and TPO.758 compared to vehicle control; the stimulation index decreased from 3.21 to 1.53 for GAD.492 (p<0.01) (FIG. 8A) and from 6.39 to 3.11 for TPO.758 (p<0.05) (FIG. 8B). S53 also suppressed T-cell proliferation induced by Tg.1571, although not statistically significant (FIG. 9C). S53 did not significantly affect the free T4 levels or antibody levels against mouse Tg (data not shown).

In summary, it has been shown that cepharanthine (S53) blocks HLA-DR3 in vitro, ex vivo, and in vivo, and that the blocking HLA-DR3 by cepharanthine suppresses autoreactive T-cell activation against islet and thyroid antigens. While the results show that cepharanthine was active in APS3v (T1D+AITD), our data also indicates that cepharanthine will also be active against T1D that is triggered in individuals possessing the HLA-DR3 gene, which is up to 30-40% of T1D patients. Cepharanthine can thus be used in the treatment of two subsets of T1D patients: (1) T1D patients that also have AITD (APS3v) and carry the HLA-DR3 allele genotype; and (2) T1D patients that are characterized by high GAD antibodies and carry the HLA-DR3 allele genotype.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of treating and/or preventing type 1 diabetes in a subject in need thereof comprising administering to the subject a therapeutically effective amount of cepharanthine or a pharmaceutically acceptable salt, solvate, or derivative thereof, wherein the subject has a human leucocyte antigen class II, DR3 allele (HLA-DR3) genotype.

2. The method of claim 1, further comprising, prior to administering, screening the subject for the HLA-DR3 allele genotype.

3. The method of claim 1, wherein the subject further has autoimmune polyglandular syndrome type 3 variant.

4. The method of claim 1, wherein the subject further comprises a high level of serum glutamic acid decarboxylase antibody.

5. The method of claim 1, further comprising administering a therapeutically effective amount of an additional active agent to the subject.

6. The method of claim 5, wherein the additional active agent comprises Teplizumab, anti-thymocyte globulin, insulin, an amylinomimetic drug, or a combination thereof.

7. The method of claim 5, wherein the additional active agent comprises a short-acting insulin, a rapid-acting insulin, an intermediate-acting insulin, a long-acting insulin, a combination insulin, pramlintide, or a combination thereof.

8. The method of claim 1, wherein the subject is a human subject.

9. A method of treating and/or preventing autoimmune polyglandular syndrome type 3 variant in a subject comprising administering to the subject a therapeutically effective amount of cepharanthine or a pharmaceutically acceptable salt, solvate, or derivative thereof, wherein the subject has a leucocyte antigen class II, DR3 allele (HLA-DR3) genotype.

10. The method of claim 9, further comprising, prior to administering, screening the subject for the HLA-DR3 allele genotype.

11. The method of claim 9, further comprising administering an effective amount of an additional active agent to the subject.

12. The method of claim 11, wherein the additional active agent comprises Teplizumab, anti-thymocyte globulin, insulin, an amylinomimetic drug, or a combination thereof.

13. The method of claim 11, wherein the additional active agent comprises a short-acting insulin, a rapid-acting insulin, an intermediate-acting insulin, a long-acting insulin, a combination insulin, pramlintide, or a combination thereof.

14. The method of claim 9, wherein the subject is a human subject.

15. The method of claim 9, wherein the subject has a high level of serum glutamic acid decarboxylase antibody.

16. A method of treating and/or preventing type 1 diabetes in a subject having type 1 diabetes or having a high risk for type 1 diabetes, the method comprising:
determining that the subject has a human leucocyte antigen class II, DR3 allele (HLA-DR3) genotype; and
administering to the subject a therapeutically effective amount of cepharanthine, or a pharmaceutically acceptable salt, solvate, or derivative thereof.

17. The method of claim 16, wherein the determining comprises testing a sample from the subject to determine the human leucocyte antigen (HLA) genotype of the subject.

18. The method of claim 16, further comprising testing a serum sample from the subject for the presence of glutamic acid decarboxylase antibody.

19. The method of claim 16, further comprising determining that the subject comprises high levels of serum glutamic acid decarboxylase antibody.

20. The method of claim 1, comprising administering cepharanthine to the subject.


SEQUENCE LISTING (4 sequences)

SEQ ID NO: 1 - protein, 27 aa, synthetic construct
  REGION 1..27: note = Tg.1571
EKVPESKVIFDANAPVAVRSKVPDSEF

SEQ ID NO: 2 - protein, 19 aa, synthetic construct
  REGION 1..19: note = GAD.492
REGYEMVFDGKPQHTNVCF

SEQ ID NO: 3 - protein, 19 aa, synthetic construct
  REGION 1..19: note = TPO.758
ESGRRVLVYSCRHGYELQG

SEQ ID NO: 4 - protein, 19 aa, synthetic construct
  REGION 1..19: note = APO
IPDNLFLKSDGRIKYTLNK